(19) United States
(12) Patent Application Publication
(10) Pub. No.: US 2022/0395628 A1
(43) Pub. Date: Dec. 15, 2022

(54) Manifold For Medical Waste Collection System

(71) Applicant: Stryker Corporation, Kalamazoo, MI (US)

(72) Inventor: Michael Zollinger, Chelsea, MI (US)

(73) Assignee: Stryker Corporation, Kalamazoo, MI (US)

(21) Appl. No.: 17/776,162

(22) PCT Filed: Nov. 6, 2020
(86) PCT No.: PCT/US2020/059402

§ 371 Date: May 11, 2022

Related U.S. Application Data
(60) Provisional application No. 62/933,587, filed on Nov. 11, 2019.

(51) Int. Cl. A61M 1/00

(52) U.S. Cl. CPC A61M 1/79; A61M 2205/7545

(57) ABSTRACT

A manifold configured to be inserted into a receiver of a medical waste collection system for filtering medical waste, including fluid, received under the influence of a vacuum provided by the medical waste collection system. The manifold includes a housing defining a manifold volume and an outlet opening near an upper aspect of the housing. The manifold further includes a filter element and a baffle wall disposed within the manifold volume. The baffle wall is arranged to define a sump region of the manifold volume, which is positioned below a lowermost aspect of the outlet opening such that fluid in the suction path is pulled from below the outlet opening, thereby reducing a level of fluid retained within the manifold when the manifold is removed from the receiver.

### PRIORITY CLAIM

This application claims priority to and all the benefits of U.S. Provisional Patent Application No. 62/933,587, filed Nov. 11, 2019, the entire contents of which are hereby incorporate by reference.

### BACKGROUND

A byproduct of some surgical procedures is the generation of liquid, semisolid, and/or solid waste material. The liquid waste material may include bodily fluids and irrigating solution(s) at the surgical site, and the solid and semisolid waste material may include bits of tissue and pieces of surgical material(s). The medical waste, regardless of its phase, is preferably collected so it neither fouls the surgical site nor becomes a biohazard in the medical suite in which the procedure is being performed.

The medical waste may be removed from the surgical site through a suction tube under the influence of a vacuum provided by a suction source. One exemplary medical waste collection system is sold under the tradename NEPTUNE by Stryker Corporation (Kalamazoo, Mich.) with certain versions of the medical waste collection system disclosed in commonly owned United States Patent Publication No. 2005/0171495, published Aug. 4, 2005, International Publication No. WO 2007/070570, published Jun. 21, 2007, and International Publication No. WO 2014/066337, published May 1, 2014, the entire contents of each are incorporated herein by reference.

A manifold may be provided that facilitates interfacing the suction tube with the medical waste collection system and may include a filter element that facilitates filtering the waste material to avoid clogging or compromise of internal components of the medical waste collection system. The manifold may be removably coupled with the medical waste collection system before or during the procedure and removed following the procedure to dispose of trapped waste material from the filter element. As mentioned above, the medical waste may be a biohazard in the medical suite and, as such, reliable containment of the medical waste avoids cross-contamination. Preventing the escape of waste material from the manifold remains an area of particular interest and development.

### SUMMARY

A manifold for filtering medical waste including fluid received from a suction tube under the influence of a vacuum provided by a medical waste collection system. The medical waste collection system includes a receiver defining an opening. The manifold is configured to be inserted into the opening in a proximal direction and removed in a distal direction opposite the proximal direction. The manifold includes a housing defining a manifold volume and an outlet opening in fluid communication with the manifold volume. The housing includes a rim defining the outlet opening positioned near an upper aspect of the housing when the manifold is oriented for insertion into the opening of the receiver. A seal is coupled to the rim and includes a seal body shaped to cover the outlet opening. The seal is configured to prevent egress of the fluid when the manifold is removed from the receiver. A filter element is disposed within the manifold volume. An inlet fitting is configured to receive the suction tube such that a suction path is established from the suction tube to the outlet opening across the filter element. A baffle wall is disposed within the manifold volume. The baffle wall and the housing define a sump region of the manifold volume that is positioned below a lowermost aspect of the seal such that the fluid in the suction path is pulled from below the seal, thereby reducing a level of the fluid retained within the manifold.

A manifold for filtering medical waste including fluid received from a suction tube under the influence of a vacuum provided by a medical waste collection system. The medical waste collection system includes a receiver defining an opening. The manifold is configured to be inserted into the opening in a proximal direction and removed in a distal direction opposite the proximal direction. The manifold includes a housing defining a manifold volume and an outlet opening in fluid communication with the manifold volume. The housing includes a body portion extending to a distal aspect, and a leg extending proximally from the distal aspect. The leg includes a rim defining the outlet opening. A seal is coupled to the rim and includes seal body shaped to cover the outlet opening and prevent egress of the fluid when the manifold is removed from the receiver. A filter element is disposed within the manifold volume. An inlet fitting is configured to receive the suction tube such that a suction path is established from the suction tube to the outlet opening across the filter element. A baffle wall is disposed within the manifold volume and spaced apart from the distal aspect. The baffle wall and the distal aspect cooperate to reduce a cross-sectional area of the suction path near the outlet opening to less than a cross-sectional area of the suction path opposite the baffle wall.

A manifold for filtering medical waste including fluid received from a suction tube under the influence of a vacuum provided by a medical waste collection system. The medical waste collection system includes a receiver defining an opening. The manifold is configured to be inserted into the opening in a proximal direction and removed in a distal direction opposite the proximal direction. The manifold includes a housing defining a manifold volume and an outlet opening in fluid communication with the manifold volume. The housing includes a rim defining the outlet opening. A filter element includes a basket having a base. The filter element defines a sump region of the manifold volume between the base and the outlet opening. An inlet fitting is configured to receive the suction tube such that a suction path is established from the suction tube to the outlet opening across the filter element. A baffle wall is coupled to the base of the basket. The baffle wall arranged to redirect the suction path.

A manifold for filtering medical waste including fluid received from a suction tube under the influence of a vacuum provided by a medical waste collection system. The medical waste collection system includes a receiver defining an opening. The manifold is configured to be inserted into the opening in a proximal direction and removed in a distal direction opposite the proximal direction. The manifold includes a housing defining a manifold volume and an outlet opening in fluid communication with the manifold volume. A filter element is disposed within the manifold volume. The filter element includes a base wall and at least an upper and lower wall extending distally from the base wall. A baffle wall is disposed within the manifold volume and spaced apart proximally from the base wall to define a gap. An inlet fitting configured to receive the suction tube. A first suction path is established from the suction tube to the outlet opening across the lower wall of the filter element. A second suction path is established from the suction tube to the outlet opening across the upper wall of the filter element, through the gap.

A manifold for filtering medical waste including fluid received from a suction tube under the influence of a vacuum provided by a medical waste collection system. The medical waste collection system includes a receiver defining an opening. The manifold is configured to be inserted into the opening in a proximal direction and removed in a distal direction opposite the proximal direction. The manifold includes a housing defining a manifold volume and an outlet opening in fluid communication with the manifold volume. The housing includes a rim defining having a width greater than a height such that the outlet opening has a non-circular profile. A filter element is disposed within the manifold volume. An inlet fitting is configured to receive the suction tube such that a suction path is established from the suction tube to the outlet opening across the filter element. A baffle wall is disposed within the manifold volume and spaced apart from the housing to define an outlet region. Opposing portions of the baffle wall and the housing defining the outlet region are shaped such that the outlet region has a non-circular profile at least substantially similar to the non-circular profile as the outlet opening.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 is a perspective view of a medical waste collection system with two manifolds configured to be removably inserted into a respective receiver of the medical waste collection system. Two suction tubes are configured to be removably coupled to each of the two manifolds.

FIG. 2 is a perspective view of the medical waste collection system and the manifolds of FIG. 1 with a front cover of the medical waste collection system removed to show each of the receivers coupled to a respective waste container of the medical waste collection system.

FIG. 3 is a perspective view of the manifold and the receiver with the manifold oriented for insertion into an opening of the receiver.

FIG. 4 is a perspective view of the manifold of FIG. 3 showing a housing.

FIG. 5 is a cross-sectional perspective view of the manifold taken along line 5-5 of FIG. 4 showing a body portion of the housing, a filter element, and a seal.

FIG. 6 is an oblique cross-sectional view of the manifold of FIG. 5 showing a baffle wall and an interior surface of the body portion.

FIG. 7 is another cross-sectional view of the manifold showing portions of a manifold volume.

FIG. 8 is an exploded perspective view of the manifold of FIG. 4.

FIG. 9 is a rear perspective view of the housing.

FIG. 10 is a cross sectional view of the housing taken along line 10-10 in FIG. 16.

FIG. 11 is a rear elevation view of the housing.

FIG. 12 is a side elevation view of the housing.

FIG. 13 is a simplified cross-sectional side view of the housing of FIG. 10 showing portions of the manifold volume and a suction path.

FIG. 14 is a close-up detailed side elevation view of a portion of the housing.

FIG. 15 is a bottom plan view of the housing.

FIG. 16 is a top plan view of the housing.

FIG. 17 is a front perspective view of the filter element of FIG. 5.

FIG. 18 is a rear perspective view of the filter element with a portion of the baffle wall in phantom showing a post.

FIG. 19 is a top plan view of the filter element.

### DETAILED DESCRIPTION

With reference to the Figures, wherein like numerals indicate like parts throughout the several views, the present disclosure includes a medical waste collection system 100 for collecting waste material generated during a medical procedure, and more particularly a surgical procedure. The medical waste collection system 100 collects the waste material and/or stores the waste material until it is necessary or desired to off-load and dispose of the waste material. The medical waste collection system 100 may be transported to and operably coupled with a docking station through which the waste material is emptied.

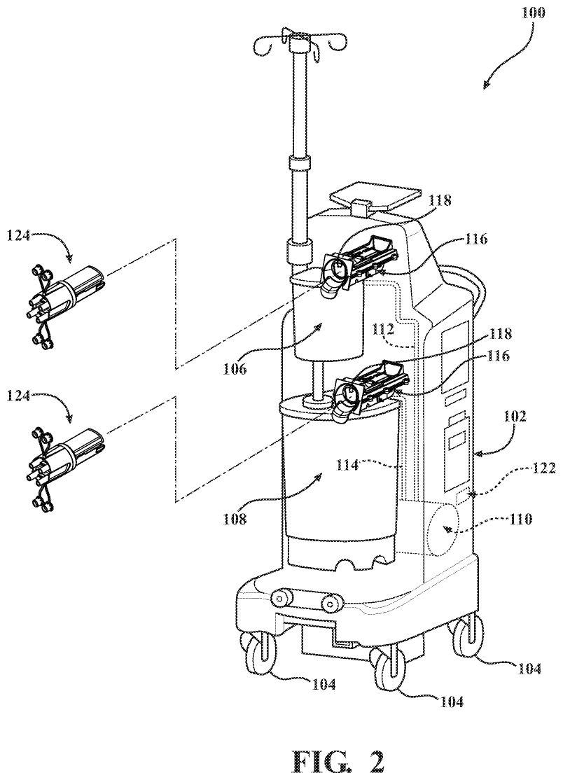

The medical waste collection system 100 may include a base 102 and wheels 104 for moving the system 100 along a floor surface within a medical facility. The medical waste collection system 100 includes at least one waste container 106, 108 defining a waste volume for collecting and storing the waste material. FIG. 2 shows a first waste container 106 arranged above a second waste container 108 having a relatively greater or larger volume than the first waste container 106. A vacuum pump 110 (in phantom) is supported on the base 102 and configured to draw suction on one or both of the first and second waste containers 106, 108 through one or more vacuum lines 112, 114. At least one vacuum regulator (not shown) may also be supported on the base 102 and in fluid communication with the vacuum pump 110 and the waste container(s) 106, 108. The vacuum regulator(s) are configured to regulate a level of the suction drawn on the waste container(s) 106, 108. Suitable construction and operation of several subsystems of the medical waste collection system 100 are disclosed in aforementioned commonly owned United States Patent Publication No. 2005/0171495, International Publication No. WO 2007/070570, and International Publication No. WO 2014/066337. Suitable construction and operation of several subsystems of the medical waste collection system 100 may also be disclosed in commonly owned International Publication No. WO 2017/112684, published Jun. 29, 2017, the entire contents of which are hereby incorporated by reference.

The medical waste collection system 100 includes at least one receiver 116 supported on the base 102. In a most general sense, the receiver(s) 116 define an opening 118 sized to removably receive at least a portion of a manifold 124 in a manner to be described throughout the present disclosure. FIG. 2 shows two receivers 116 with each of the receivers 116 associated with a respective one of the first and second waste containers 106, 108. Alternatively, a single receiver and/or a single manifold may be provided. The receiver(s) 116 include a suction inlet configured to be arranged in fluid communication with the respective one of the waste containers 106, 108. A suction path 122 may be established from suction tube(s) 120 to the waste containers 106, 108 through the manifold(s) 124, which is removably inserted into the receiver(s) 116. As will be discussed in further detail below, the vacuum generated by the vacuum pump 110 is drawn on the suction path 122 via the suction tube(s) 120 such that waste material at the surgical site is drawn through the manifold(s) 124, through the suction inlet and into the waste container(s) 106, 108. Suitable construction and operation of the receiver 116 of the medical waste collection system 100 are disclosed in commonly owned U.S. patent application Ser. No. 16/383,218.

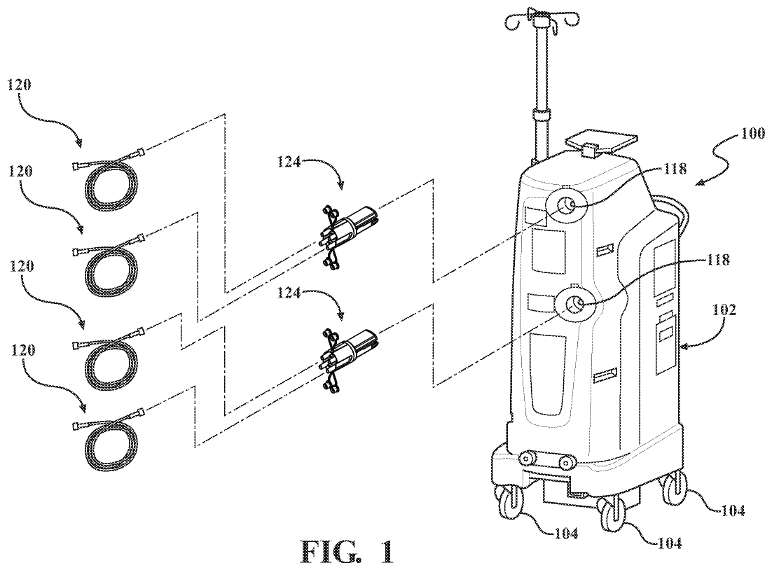

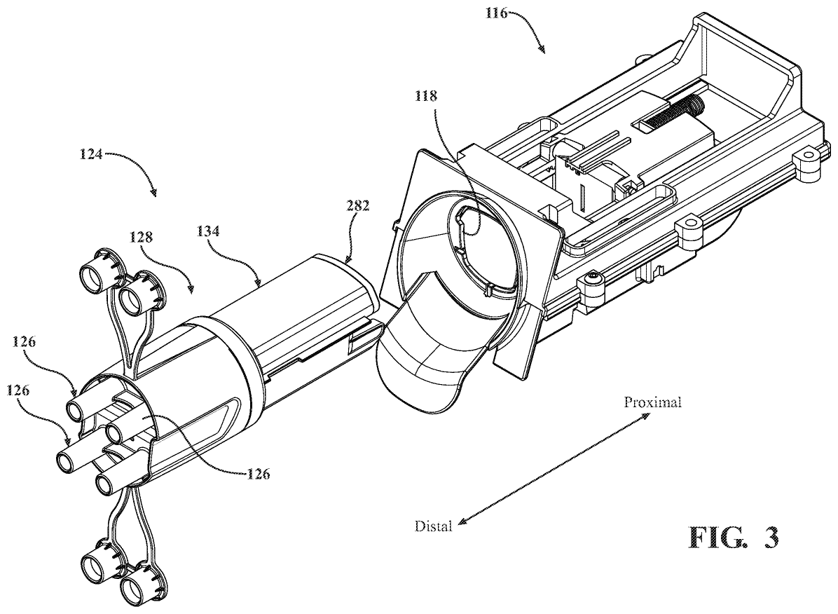

Referring to FIG. 3, the manifold 124 is shown in a decoupled operative position in which the manifold 124 is separated or spaced apart from the receiver 116. FIG. 3 may be representative of the manifold 124 prior to insertion into the receiver 116 and/or after removal of the manifold 124 from the receiver 116. The manifold 124 is configured to be inserted into the receiver 116 through the opening 118, and the suction tube(s) 120 are coupled to inlet fitting(s) 126 of the manifold 124. The resulting arrangement is schematically reflected in FIG. 1 in which two suction tubes 120 are coupled to two of four inlet fittings 126 of each of the manifolds 124. Any number of inlet fitting(s) are contemplated, and it is further contemplated that the suction tube(s) 120 may be integral with the housing 128. The aforementioned suction path 122 is established, and an instrument (not shown) coupled to an end of the suction tube(s) 120 opposite the manifold(s) 124 may be directed to the surgical site to collect the waste material under the influence of the vacuum provided by the vacuum pump 110.

With further reference to FIG. 3, the manifold 124 includes a housing 128. As will be described in further detail below, the housing 128 may be at least similar to that described in commonly owned U.S. Pat. No. 10,471,188, issued Nov. 12, 2019, the entire contents of which are hereby incorporated by reference, with description abbreviated in the present disclosure not to be construed as limiting to either application. The housing 128 defines a manifold volume 130 having an interior surface 131. An inlet fitting 126 and an outlet opening 242 are further defined in the housing and in fluid communication with the manifold volume 130, in certain configurations. The inlet fitting 126 is configured to receive the suction tube 120 such that the suction path 122 is established from the suction tube 120 to the outlet opening 242 across a filter element 174, as will be described in further detail below. The housing 128 may be considered any external structure or component of the manifold 124, and more particularly any structure or component that at least partially defines the manifold volume 130.

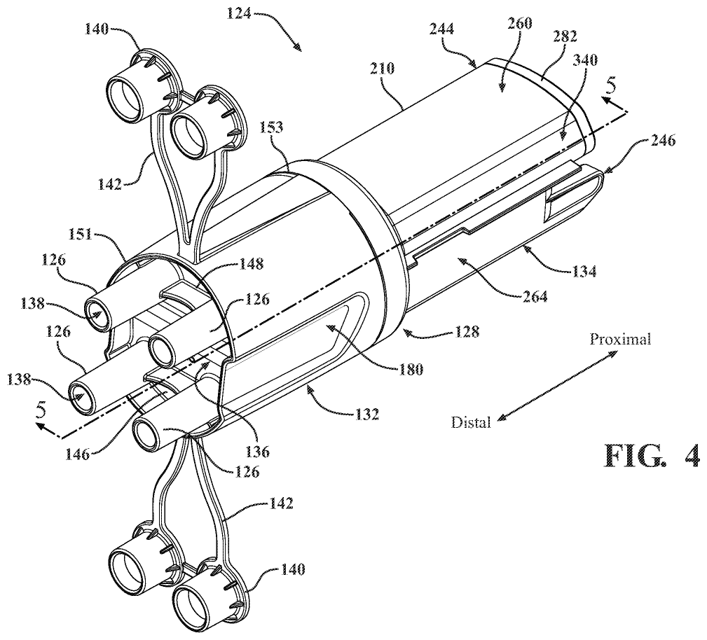

As shown in FIGS. 3 and 4, the manifold 124 may comprise a head 132 coupled to a trunk 134 to at least partially form the housing 128. The head 132 is positioned distal to the trunk 134 when the manifold 124 is oriented for insertion into the opening 118 of the receiver 116, as shown in FIG. 3. As used throughout the present disclosure, the terms “distal” and “proximal” may refer to the respective directions identified in arrows of FIGS. 3-6, 8, and 9. In another convention, the term “distal” may refer to a direction generally away from the receiver 116, and the term “proximal” may refer to a direction generally towards the receiver 116. In still another convention, the term “distal” may refer to a direction generally towards a front of the manifold 124 and towards the surgical site, and the term “proximal” refers to a direction generally towards a rear of the manifold 124 (when the manifold 124 is inserted into the receiver 116) and away from the surgical site. In an alternative to the multi-piece construction including the head 132 and the trunk 134, the housing 128 of the manifold 124 may be of unitary or monolithic construction. The head 132 (or any other portion of the housing 128) may include and/or be coupled to the inlet fitting(s) 126. The inlet fitting(s) 126 may extend distally from a crown 136 to define a distal end of the manifold 124. Alternatively, the inlet fitting(s) 126 may be coupled to a different structure separate from the housing 128 (i.e., not directly coupled to the head 132) with the inlet fitting(s) 126 being in fluid communication with the outlet opening 242, to be described, to establish the suction path 122. It is further contemplated that any features described as being a part of the head 132 may alternatively be a part of the trunk 134, and any features described as being a part of the trunk 134 may alternatively be a part of the head 132.

In certain implementations, the housing 128 may merely provide the structural support for the certain various components without defining the inlet fitting(s) 126 or the outlet opening 242. In other words, the housing 128 may not define the manifold volume 130 but instead support certain structures described below, including but not limited to arm(s) 284, catch(es) 254, a spine 300, and/or lock element(s) 306. For example, an arrangement in which a volume and/or outlet opening is defined by a structure separate from a housing is described in commonly-owned U.S. application Ser. No. 16/679,922, filed Nov. 11, 2019, the entire contents are hereby incorporated by reference.

FIGS. 3 and 4 show four inlet fittings 126 extending distally from the crown 136 in a parallel arrangement, according to one implementation. Each of the inlet fittings 126 may define an inlet bore 138. The manifold 124, therefore, is configured to be removably coupled with four of the suction tubes 120 to be simultaneously operable with operation of the medical waste collection system 100. Here, each of the inlet fittings 126 is arranged in parallel in the suction path 122. In this way, waste material that has been drawn through any of the suction tubes may enter the manifold 124 through any one of the respective inlet bores 138. While each inlet fitting 126, and thus each inlet bore 138, are distinct from each other, the inlet bores 138 collectively form an inlet portion of the manifold volume 130.

Should one or more of the inlet fittings 126 not be removably coupled with the suction tube 120, a cap 140 may be removably coupled with a distal portion of the inlet fitting(s) 126 to seal a respective one of the inlet bores 138 from fluid communication with the ambient. The resulting arrangement prevents ambient air from being drawn into the inlet bores 138 under the influence of the vacuum when the respective inlet fitting(s) 126 are not intended for use for at least a portion of the surgical procedure. The vacuum may be directed through the inlet fitting(s) 126 to which the suction tube(s) 120 are coupled without compromise, thereby providing improved control of the vacuum level at the surgical site. The cap(s) 140 may be coupled to the housing 128 with one or more tethers 142 extending outwardly from the head 132, and pairs of the caps 140 may be coupled to one another such that the pairs of the caps 140 may be coupled to and decoupled from respective pairs of the inlet fittings 126 in tandem. Alternatively, the caps 140 may be provided separately, and/or the tether(s) 142 may be removably coupled to the head 132.

The crown 136 shown in FIG. 4 may include a lower face 146 and an upper face 148 each from which a pair of the inlet fittings 126 extend distally. The lower face 146 is positioned below the upper face 148 when the manifold 124 is oriented for insertion within the opening 118 of the receiver 116. The lower face 146 may be positioned more distal to the upper face 148 such that, owing to a length of the inlet fittings 126 being equal, a lower pair of the inlet fittings 126 extend more distally than an upper pair of the inlet fittings 126.

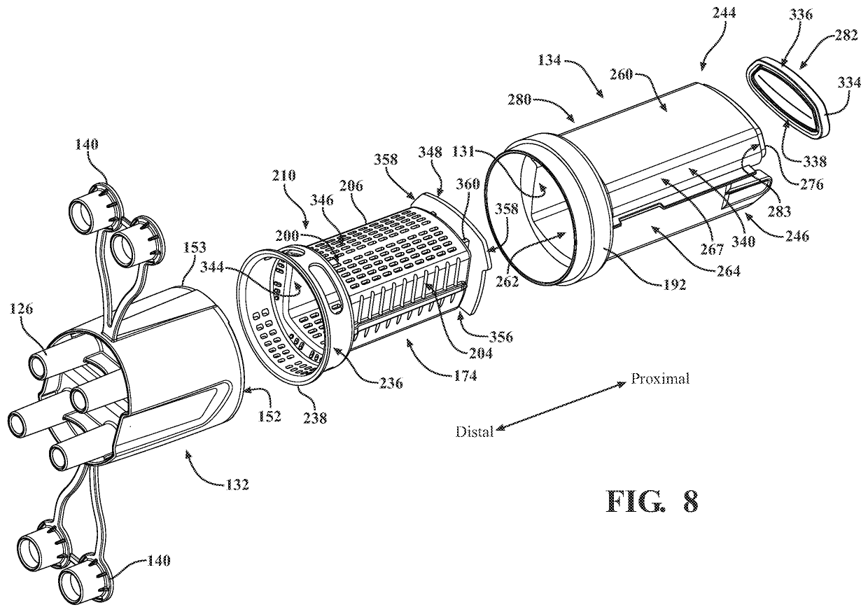

The head 132 is engageable with the body portion 210 such that the suction tubes 120 are in fluid communication with the manifold volume 130. The head 132 may include at least one side 150, which may extend between a distal rim 151 opposite a proximal rim 153. The distal rim 151 may be positioned slightly distal to the crown 136 such that the crown 136 is recessed. The side 150 may be considered a singular surface that is generally cylindrical in shape or plural surfaces arranged in any suitable geometry. Illustrated in the exploded view of FIG. 8, an inner or proximal surface of the crown 136 and an inner surface of the side 150 may cooperate to define a cavity 152 that may define at least a portion of the manifold volume 130. In such an arrangement, the inlet bore(s) 138 are in fluid communication with the cavity 152, and thus the manifold volume 130.

In certain implementations, the head 132 and the trunk 134 are removably coupled to one another such that the manifold volume 130 of the trunk 134 may be accessed through a distal opening at least partially defined by a neck 192 of the trunk 134, as shown in FIG. 8. The head 132 and the trunk 134 may be positively retained with complementary features coupled to the head 132 and the trunk 134. In one example, the manifold may comprise a bayonet type coupler. More specifically, the head 132 may include at least one key or head coupler configured to be removably engaged with at least one keyway or trunk coupler of the trunk 134. The key and the keyway may be secured by inserting the key into the keyway and rotating the head 132 relative to the trunk 134. Alternative arrangements of the head coupler and the trunk coupler may be implemented to effect attachment of the head 132 and the trunk 134. For example, the head 132 may comprise internal threads and the trunk 134 may comprise corresponding external threads such that the head 132 and the trunk 134 may be screwed together. Alternatively, only one of the head 132 and the trunk 134 may be configured with external threads and a nut may be used to bring sealing faces into sealing contact and removably couple the head 132 to the trunk 134. When it is desired to decouple the head 132 from the trunk 134, the aforementioned method steps are reversed.

In certain implementations, the head 132 and the trunk 134 are rigidly connected through a suitable joining process, for example, spin welding, solvent bonding, adhesives, mechanical fastening, and the like. As previously mentioned, the housing 128 may be of unitary or monolithic construction such that there is no discrete head and trunk. Suitable manufacturing processes for forming the housing 128 may include injection molding, three-dimensional printing, computer numerical control (CNC) machining, polymer casting, vacuum forming, blow molding, among others. Suitable materials for forming the housing 128 may include polymers, composites, metals, ceramics, and combinations thereof. The materials may have sufficient anticorrosive properties or coatings to avoid degradation when exposed to the waste material, and sufficient mechanical properties to maintain integrity under the vacuum levels to be provided by the medical waste collection system 100. The polymers of polyethylene, polypropylene, polyvinyl chloride, polyethylene terephthalate (PET, PETE), polystyrene, polycarbonate, and poly(methyl methacrylate) may be particularly well suited for the manifold 124 in low-cost and disposable implementations.

The removable coupling between the head 132 and the trunk 134 provides access to the manifold volume 130 within which a filter element 174 may be disposed. Among other advantages, accessing the filter element 174 may allow the user to retrieve waste material collected within the filter element 174, most notably a polyp or tissue sample, for further examination and processing during certain surgical procedures. Commonly owned International Publication No. WO 2013/090579, published Jun. 20, 2013, the entire contents of which is hereby incorporated by reference, discloses a manifold including a tissue trap for collecting the polyp or the tissue sample. In certain implementations, the manifold 124, including the head 132, may include further features to facilitate collection of tissue sample(s).

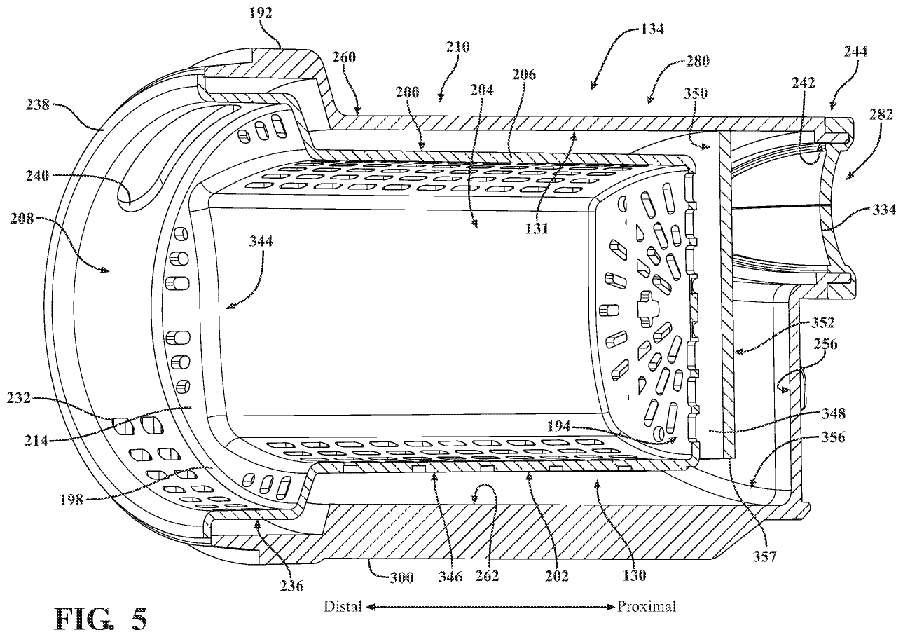

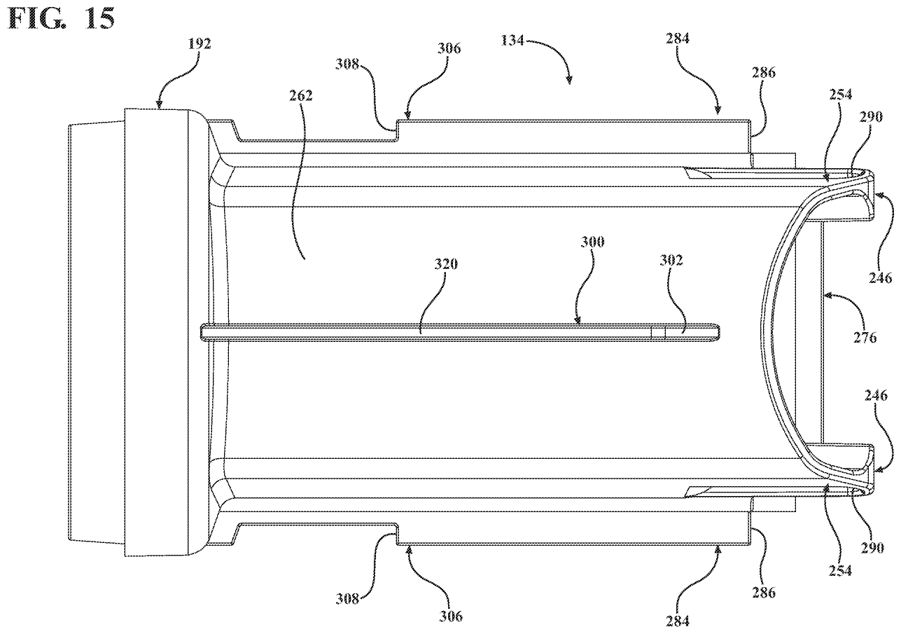

Referring now to FIGS. 5-16, one implementation of the housing 128 of the manifold 124 is shown comprising a body portion 210. Here, the body portion 210 comprises a first leg 244 and a second leg 246, the first leg 244 is positioned above the second leg 246 with certain structures located in a corresponding manner. It should be understood that counterpart components on the receiver 116 may be modified in an appropriate manner to permit the trunk 134 to be inserted into and removed from the receiver 116 in the manners previously described. Further, it should be understood that, while discussed in the context of the trunk 134, which may be integrated with or coupled to any suitable head, for example the head 132 described above, the disclosure may be applicable to the housing 128 and/or the manifold 124 more generally.

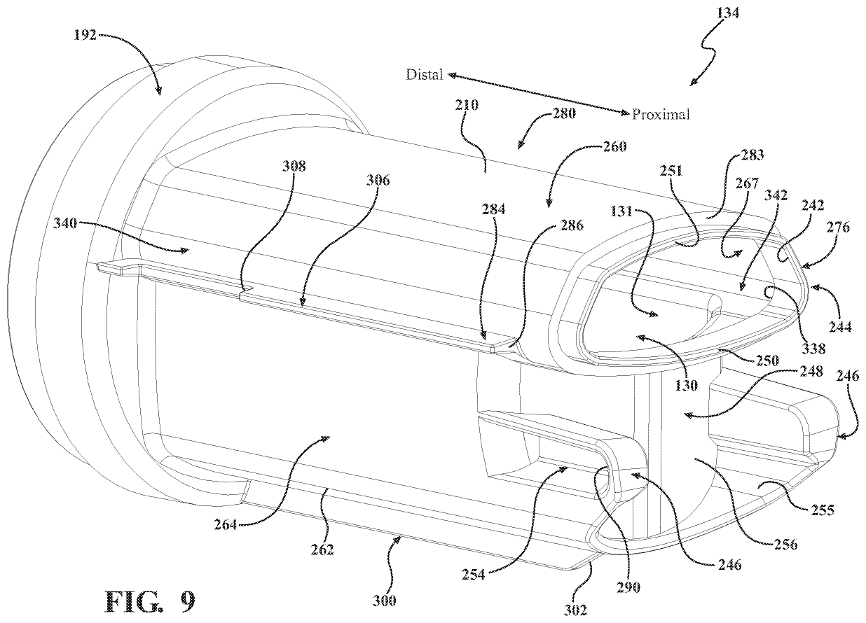

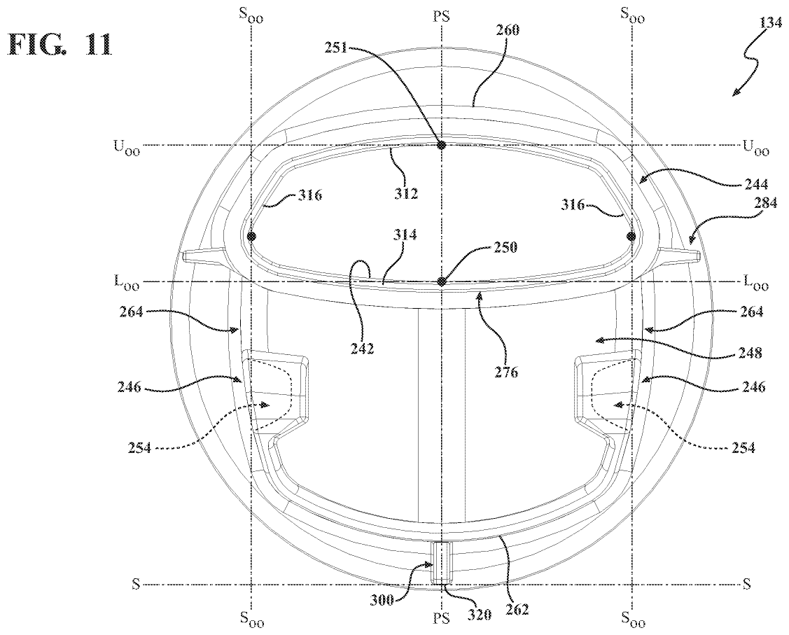

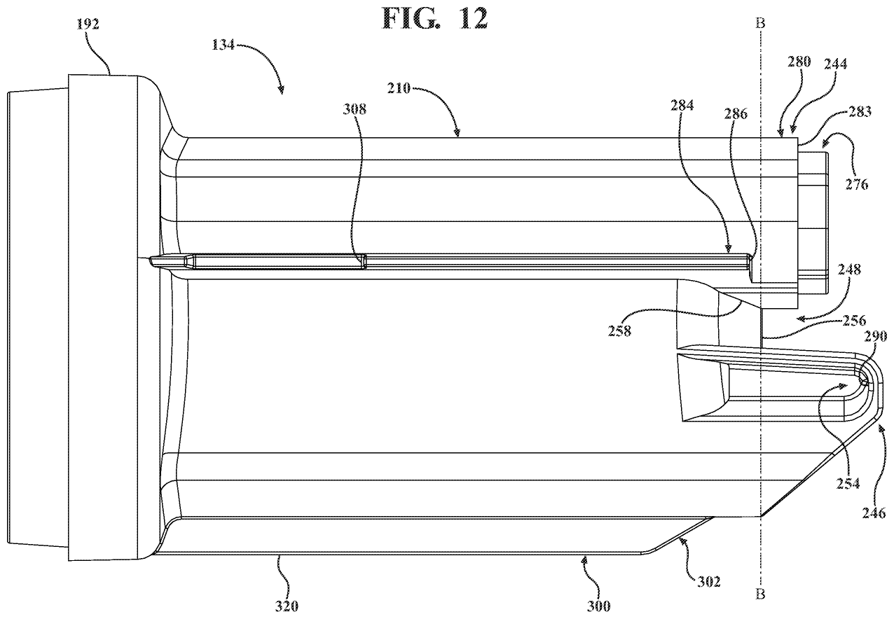

Shown in FIG. 9, the trunk 134 includes the body portion 210, the first leg 244, and/or the second leg 246, which cooperate to define at least a part of the manifold volume 130. The first leg 244 and/or the second leg 246 may extend from the body portion 210, and more particularly one or both of the first and second legs 244, 246 may extend proximally from the body portion 210. Here, each of the first and second legs 244, 246 extend proximally from a proximal wall 228 of the housing 128. The first and second legs 244, 246 may be spaced apart from one another to at least partially define a void 248, as best shown in the perspective view of FIG. 9 and the elevation views of FIGS. 11, 12 and 14. For convention, the vertical plane perpendicular to the proximal-to-distal direction and extending through a proximal-most point of the distal aspect 256 may be considered the boundary (B) separating the body portion 210 and the first and/or second legs 244, 246, as identified in FIG. 12. A portion of the trunk 134 distal to the boundary (B) may be considered the body portion 210, and upper and lower portions of the trunk 134 proximal to the boundary (B) may be considered the first and second legs 244, 246, respectively.

As mentioned, the first leg 244 is positioned above the second leg(s) 246 when the manifold 124 is oriented for insertion into the opening 118 of the receiver 116. The trunk 134 may include an upper wall 260, a lower wall 262, and opposing sides 264. The opposing sides 264 and the lower wall 262 may cooperate to form at least a portion of the second leg(s) 246, and the opposing sides 264 and the upper wall 260 may cooperate to form at least a portion of the first leg 244 such that the first leg 244 is at least partially defined by the upper wall 260. A tubulate wall 280 at least partially defining the first leg 244 may include an upper aspect 252, the opposing sides 264, and the upper wall 260.

Cross-sectional views in FIGS. 5-7 and 10 illustrate details of the trunk 134 and the manifold volume 130. The interior surface 131 at least partially defines the manifold volume 130 and regions thereof. When the head 132 is engaged to the trunk 134 the cavity 152 of the head 132 and the interior surface 131 of the trunk 134 cooperate to define the manifold volume 130. The interior surface 131 comprises inner faces of each of the upper wall 260, the lower wall 262, and the opposing sides 264. Here, the lower wall 262 forms a floor of the manifold volume, the opposing sides 264 form generally vertical side walls, and the upper wall 260 forms a ceiling, when the manifold is oriented for insertion into the opening 118 of the receiver 116.

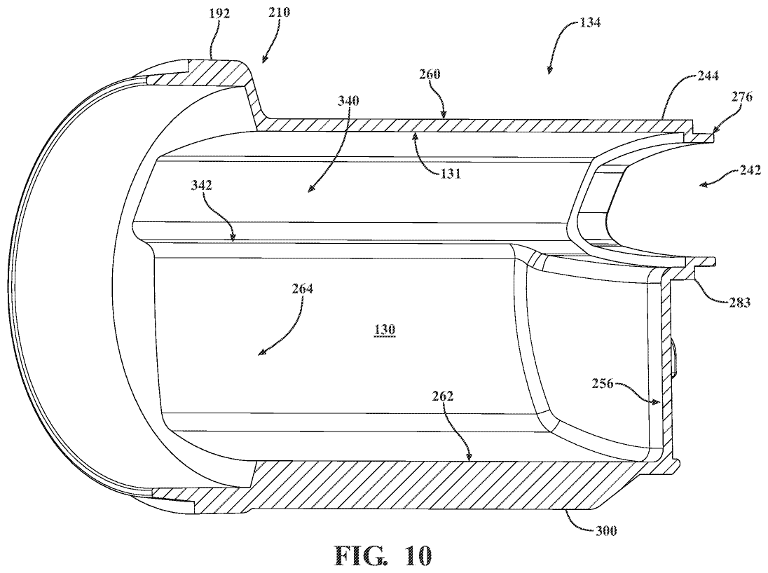

Visible in both the perspective view of FIG. 9 and the cross-sectional view of FIG. 10, each of the opposing sides 264 define a shoulder portion 340 of the housing 128. On the interior of the housing 128 the shoulder portion 340 includes a shelf 342 that extends outwardly from the vertical side walls creating a larger cross-sectional area in the upper half of the manifold volume 130. At an outer end of the shelf 342 transition walls 267 extend between the upper wall 260 and the shelf 342 to encircle the manifold volume 130. On the outside of the trunk 134 the shoulder portion 340 of the opposing sides 264 follows a similar path, extending outwardly, or horizontally, away from a vertical plane of symmetry PS. The transition walls 267 extend between the upper wall 260 and an outermost point of the shoulder portion. The exterior features of the shoulder portion 340 may serve to orient the manifold 124 when the user inserts the manifold 124 in the opening 118 of the receiver 116. As will be discussed below, the interior features of the shoulder portion 340 may serve to orient the filter element 174 during assembly of the manifold 124 and to prevent the misalignment during use.

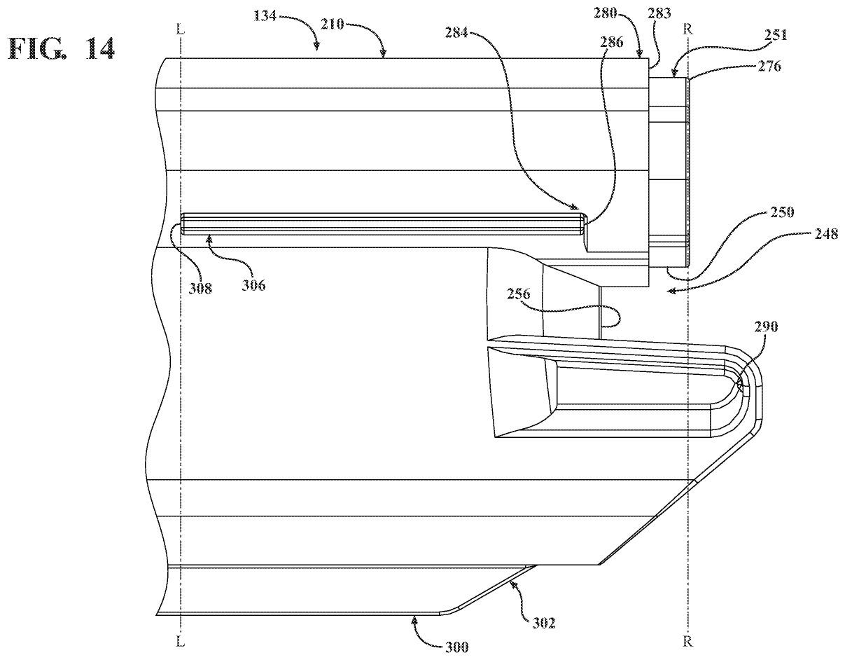

With reference to FIGS. 9, 11, 12, and 14, the trunk 134 may include the rim 276 defining the outlet opening 242. The rim 276 may be arranged on the first leg 244, and more particularly at or near a proximal end of the first leg 244. Said differently, the tubulate wall 280 at least partially defining the first leg 244 and the upper wall 260 may define the outlet opening 242. A step 283 may extend radially inward from the tubulate wall 280 at least partially defining the first leg 244 with the rim 276 extending proximally from the step 283. FIG. 14 includes the vertical plane perpendicular to the proximal-to-distal direction and extending through the rim 276, identified as (R), indicative of a proximal-to-distal location of the rim 276. The rim 276 may include a width greater or larger than a height such that the outlet opening 242 is non-circular. The tubulate wall 280 may also include a width greater or larger than a height, and the dimensions of the rim 276 may be approximately equal to the dimensions of the tubulate wall 280 such that the outlet opening 242 is shaped complementarily to and/or to approximate a cross section of the first leg 244. The rim 276 may be configured to be coupled to a seal 282, discussed below.

Best seen in FIG. 11, the outlet opening 242 may be oblong and defined by the rim 276 including an upper segment 312, a lower segment 314, and opposing side segments 316. The vertical and horizontal planes in the proximal-to-distal direction and extending through each of the opposing lateral-most points, the uppermost aspect 251, and the lowermost aspect 250 of the outlet opening 242, respectively, are labelled (S₀₀), (U₀₀), and (L₀₀), respectively. The vertical plane in the proximal-to-distal direction and bifurcating the outlet opening 242 may be the vertical plane of symmetry (PS).

The outlet opening 242 may be positioned entirely within an upper half of the trunk 134 when the manifold 124 is oriented for insertion within the opening 118 of the receiver 116. The positioning of the outlet opening 242 near the upper wall 260 within the upper half may be particularly advantageous to avoid inadvertent egress of the waste material from the manifold volume 130. In other words, the waste material that is not drawn through the seal 282 under the influence of the vacuum may descend under the influence of gravity within the manifold volume 130. The waste material may collect within a portion of the manifold volume 130 defined by the lower wall 262 and bounded proximally by the second leg(s) 246 and/or the distal aspect 256. Further, during removal of the manifold 124 including the trunk 134 from the receiver 116 the likelihood of egress of the waste material through the seal 282 is appreciably reduced, as minimal waste material may be present on a distal side of the seal 282 (having descended to a sump region 364 within the manifold volume 130).

Referring now to FIGS. 5, 6 and 8, the manifold 124 may further comprise the seal 282 to prevent egress of the waste material when the manifold 124 is removed from the receiver 116. The seal 282 comprises a seal body 334 shaped to cover the outlet opening 242 and may include an outer seal rim 328, and an inner seal rim 330 spaced apart from the outer seal rim 328 to define a groove 332. The groove 332 is shaped complementary to the shape of the rim 276, and the groove 332 is sized to snugly receive the rim 276 such that the seal 282 and the trunk 134 may be coupled to one another via interference engagement. The seal 282 is arranged such that when the rim 276 is received in the groove 332 of the seal 282, the distally oriented face of the seal 282 abuts the step 283.

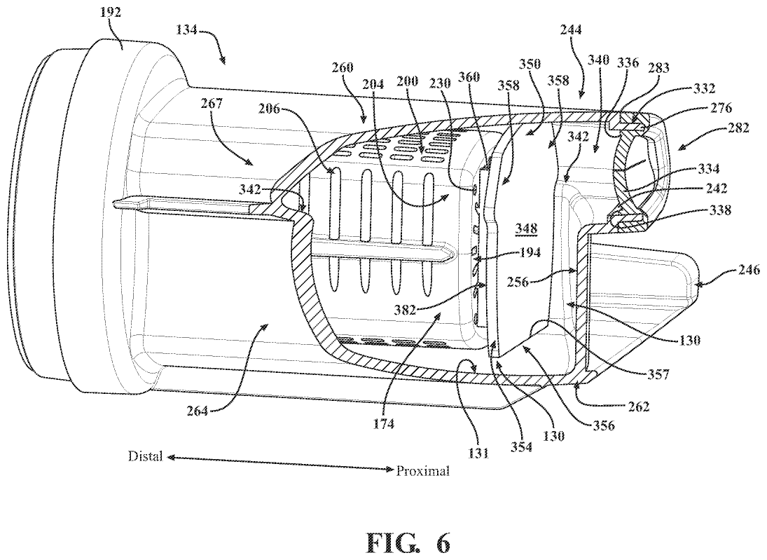

As best shown in FIGS. 5 and 6, a proximal end or edge of the inner seal rim 330 is positioned distal to a proximal end or edge of the outer seal rim 328. A depth of the outer seal rim 328 in the proximal-to-distal direction may be approximately equal to a depth of the first leg 244 proximal to the step 283. A thickness of the outer seal rim 328 may be approximately equal to a size of the step 283. With the seal 282 coupled to the rim 276, an outer surface of the outer seal rim 328 is substantially flush with the tubulate wall 280 at least partially defining the first leg 244. The arrangement may include an upper aspect of the outer seal rim 328 spaced apart from the second leg 246 and at least partially defining the void 248.

In addition to the complementary shape of the seal 282 and the rim 276, the seal 282 may include complementary features. Specifically, the seal includes an upper segment, a lower segment, and opposing side segments. An upper aspect 336 of the seal 282 is arranged near the upper aspect 252 of the first leg 244 and a lowermost aspect 338 of the seal 282 is arranged near the lowermost aspect 250 of the outlet opening 242.

Alignment of the manifold 124 during insertion into the receiver 116 may be facilitated by the inclusion of at least one arm 284 extending outwardly from the housing 128. A pair of arms 284 are illustrated throughout the Figures, but it is appreciated that a singular arm may be provided. The arms 284 may extend outwardly from at least one of the body portion 210 and the first leg 244. In other words, the arms 284 may extend away from the manifold volume 130. The arms 284 each include a proximally-directed surface 286, and the proximally-directed surfaces 286 of the arms 284 may be positioned distal to the rim 276.

The manifold 124 may include at least one catch 254, shown here as a pair of catches 254. The catches 254 may be disposed on the second leg 246, as best shown in FIGS. 9, 11, 12, and 15. The rim 276 and at least one of the catches 254 may be spaced apart from one another by the void 248. More particularly, the rim 276 on the first leg 244 may be spaced apart from the catches 254 on the second leg 246 by the void 248. Each of the catches 254 includes a distally-directed surface 290. The distally-directed surfaces 290 may be positioned proximal to the rim 276, and proximal to the proximally-directed surfaces 286 of the arms 284. Here the rim 276 on the first or upper side of the void 248, and the catches 254 on the second or lower side of the void 248. Further, the rim 276 is positioned above the catches 254 when the manifold 124 is oriented for insertion into the opening 118 of the receiver 116. Other configurations are contemplated.

Alignment of the manifold 124 and the receiver 116 may be further facilitated by a spine 300 extending outwardly from the housing 128. The spine 300 may extend outwardly from at least one of the body portion 210, the first leg 244, and/or the second leg 246. Further, the spine 300 may extend outwardly from the lower wall 262 of the trunk 134, and more particularly extend downwardly from the lower wall 262. The spine 300 includes a proximally-directed surface 302. The proximally-directed surface 302 of the spine 300 may be positioned distal to the rim 276, distal to the distally-directed surfaces 290 of the catches 254, and distal to the proximally-directed surfaces 286 of the arms 284. In certain implementations, the proximally-directed surface 302 is a ramped surface tapering towards the lower wall 262 of the housing 128 in the proximal direction to define a proximal end of the spine 300.

The spine 300 may be positioned in the lower half of the trunk 134 when the manifold 124 is oriented for insertion within the opening 118 of the receiver 116. The spine 300 may be positioned below a lowermost aspect 250 of the outlet opening 242. Further, the spine 300 may be coplanar with the plane of symmetry (PS). FIG. 11 shows the horizontal plane in the proximal-to-distal direction and extending through the downward-directed surface 320 of the spine 300, identified as (S), indicative of a downward position of the spine 300. The downward-directed surface 320 of the spine 300 is positioned below the lowermost aspect 250 of the outlet opening 242 (i.e., the plane S is below the plane L_{∞}).

The manifold 124 may further include at least one lock element 306 extending outwardly from the housing 128. The lock elements 306 may extend outwardly from at least one of the body portion 210 and the first leg 244. The lock elements 306 may extend outwardly from the trunk 134 of the housing 128, and more particularly extend laterally outward from the opposing sides 264. The lock elements 306 may include distally-directed surfaces 308. The distally-directed surfaces 308 may be positioned distal to the rim 276, distal to the distally-directed surfaces 290 of the catches 254, distal to the proximally-directed surfaces 286 of the arms 284, and distal to the proximally-directed surface 302 of the spine 300.

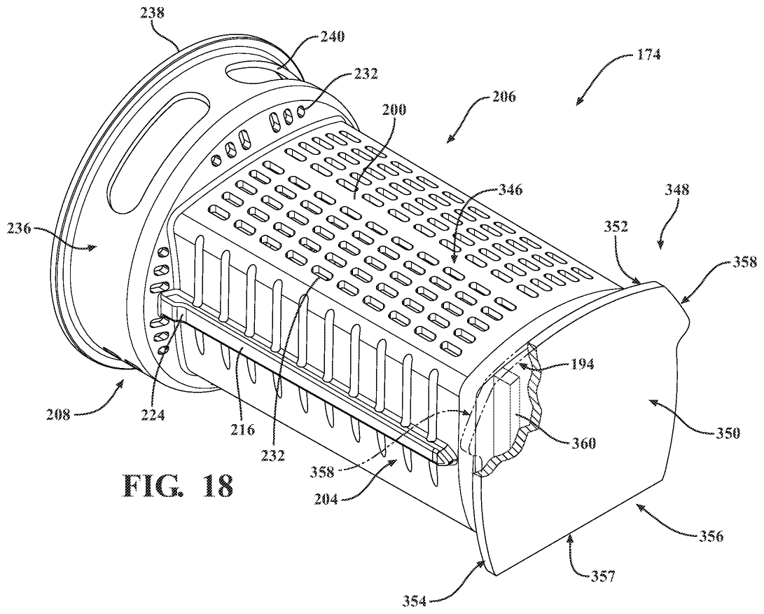

Referring now to FIGS. 4 and 16-18, the filter element 174 is disposed within the manifold volume 130 and arranged in the suction path 122 between the inlet fitting(s) 126 and the outlet opening 242. The filter element 174, in a broadest sense, includes structures configured to capture or collect the semisolid or solid waste material entrained within the liquid waste material being drawn through the manifold 124 under the influence of the vacuum provided by the medical waste collection system 100. The waste material is urged to flow through apertures, discussed below, in the filter element 174, which are sized to prevent the flow of solid or semisolid waste material that may damage the vacuum pump 110. The filter element 174 may include a basket 206 having a base wall 194 and least one side 196 extending distally from the base wall 194 to define a mouth 198 opposite the base wall 194. The basket 206 comprises an inner surface 344, generally defining an interior, and an outer surface 346. The inner surface 344, and by extension the interior, of the basket 206 are accessible through the mouth 198. Owing to geometry of the trunk 134, within which the filter element 174 is at least partially disposed, the filter element 174 may correspondingly include an upper wall 200, a lower wall 202, and opposing sidewalls 204 when the manifold 124 is oriented for insertion into the opening 118 of the receiver 116, each having a corresponding inner and outer surface. The opposing sidewalls 204 may extend between the upper and lower walls 200, 202, and each of the upper wall 200, the lower wall 202, and the opposing sidewalls 204 may extend distally from the base wall 194. The resulting arrangement may be considered the aforementioned basket 206 that is substantially square or rectangular in section. In certain implementations, the basket 206 may be substantially cylindrical in section, and other suitable shapes are contemplated.

The manifold 124 further comprises a baffle wall 348 disposed within the manifold volume 130 and spaced from the distal aspect 256 of the proximal wall 228 of the trunk 134. The baffle wall 348 and the housing 128 cooperate to define the sump region 364 of the manifold volume 130, discussed in further detail below. The baffle wall 348 comprises a proximally directed surface 350 and a distally directed surface 352 connected by a perimeter surface 354. The baffle wall 348 is arranged in the manifold volume 130 in an orientation that is generally parallel with the vertical plane. More specifically, the proximally directed surface 350 faces the proximal end of the manifold 124 and the distally directed surface 352 faces the distal end of the manifold 124. The perimeter surface 354 extends between the proximally directed surface 350 and the distally directed surface 352 in a generally horizontal direction and is configured to engage the interior surface 131 of the body portion 210. The baffle wall 348 is sized to engage the interior surface 131 of the body portion 210 with close tolerances such that fluid communication between the regions of the manifold volume 130 on opposite sides of the baffle wall 348 is not readily achieved other than through a baffle port 356, as will be discussed below.

Further, owing to geometry of the trunk 134, within which the filter element 174 is at least partially disposed, the baffle wall 348 may include a wing portion 358 that has a complementary profile to the shoulder portion 340, best shown in FIGS. 5 and 6. The complementary profiles of the wing portion 358 and the shoulder portion 340 are shaped such that the profile of corresponding wing portions 358 and shoulder portions 340 have a similar profile so as to be engageable in a nesting arrangement with each other. The wing portions 358 are shown here arranged near an upper aspect of the baffle wall 348 and extend outwardly. Because a width of the wing portions 358 is larger than a width of a lower portion of the baffle wall 348 the wing portions 358 facilitate a one-way alignment between the filter element 174 and the housing 128 such that relative rotation between the filter element 174 and the housing 128 is prevented.

As mentioned above, the baffle wall 348 defines a baffle port 356, which is arranged in the sump region 364 of the manifold volume 130. More specifically, the baffle port 356 is defined between the perimeter surface 354 of the baffle wall 348 and the interior surface 131 on the lower wall 262 of the body portion 210. The perimeter surface 354 of the baffle wall 348 defines an uppermost aspect 357 of the baffle port 356, which is spaced from the lower wall 262. The baffle port 356 is configured such that the uppermost aspect 357 is below the lowermost aspect 250 of the outlet opening 242 and the seal 282.

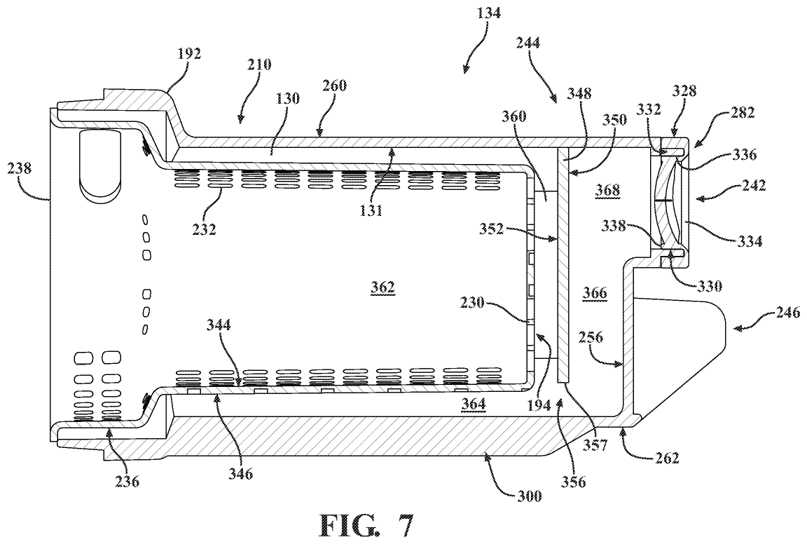

The baffle wall 348 is arranged in the manifold volume 130 to redirect the suction path 122 to a position below a lowermost aspect 250 of the outlet opening 242. Unlike the portion of the baffle wall 348 above the uppermost aspect 357 of the baffle port 356, the baffle port 356 permits the flow of waste material. Said differently, the baffle port 356 is arranged along the suction path 122 within the manifold volume 130 and permits the flow of waste material from the inlet fitting 126 and the outlet opening 242. As can be seen in FIG. 7, the cross-sectional area of the baffle port 356 is less than the cross-sectional area of the manifold volume 130 and of the outlet opening 242. Because of the reduced cross-sectional area of the baffle port 356, the suction path 122 is forced to flow through a reduced cross-sectional area.

Also shown in FIG. 7, the basket 206 of the filter element 174 is arranged in the manifold volume 130 on the distal side of the baffle wall 348, opposite of the distal aspect 256 of the proximal wall 228 of the housing 128. On the proximal side of the baffle wall 348, the baffle wall 348 and the distal aspect 256 cooperate to reduce a cross-sectional area of the suction path near the outlet opening 242. This area discussed below in connection with the suction path 122 is directed to a position below the lowermost aspect of the seal 282 to reduce the level of fluid retained in the housing 128 when the manifold 124 is removed from the receiver 116.

The filter element 174 may include a brim 208 coupled to a proximal end of the basket 206. The brim 208 extends distally from the basket 206, and may extend radially outwardly from the mouth 198 of the basket 206. The brim 208 may include an outer diameter or dimension greater or larger than an outer diameter or dimension of the basket 206. With further reference to FIG. 4, a length of the basket 206 may be such that the basket 206 is disposed within a body portion 210 of the trunk 134, and a length of the brim 208 may be such that the brim 208 is disposed within the neck 192 of the trunk 134. A step 212 extends radially inward from an inner surface of the neck 192 and/or radially outward from an inner surface of the body portion 210 to define a transition between the neck 192 and the body portion 210. The neck 192 may include an inner diameter or inner dimension greater or larger than an inner diameter or inner dimension of the body portion 210. A flared wall 214 of the filter element 174 defining a transition between the basket 206 is configured to be positioned adjacent to or in abutment with the step 212 of the trunk 134. The resulting arrangement includes the basket 206 being disposed within a portion of the manifold volume 130 defined by the body portion 210, and the brim 208 being disposed within a portion of the manifold volume 130 defined by the neck 192. Other suitable configurations are contemplated, for example, the basket 206 and/or the brim 208 may be disposed within the cavity 152 of the head 132 that may define a portion of the manifold volume 130. The brim 208 of the filter element 174 may be considered optional, and further shapes and configurations of the filter element 174 suitable for certain implementations of the manifold 124 are disclosed in commonly owned International Publication No. WO 2018/170233, filed Mar. 15, 2018, the entire contents of which are hereby incorporated by reference.

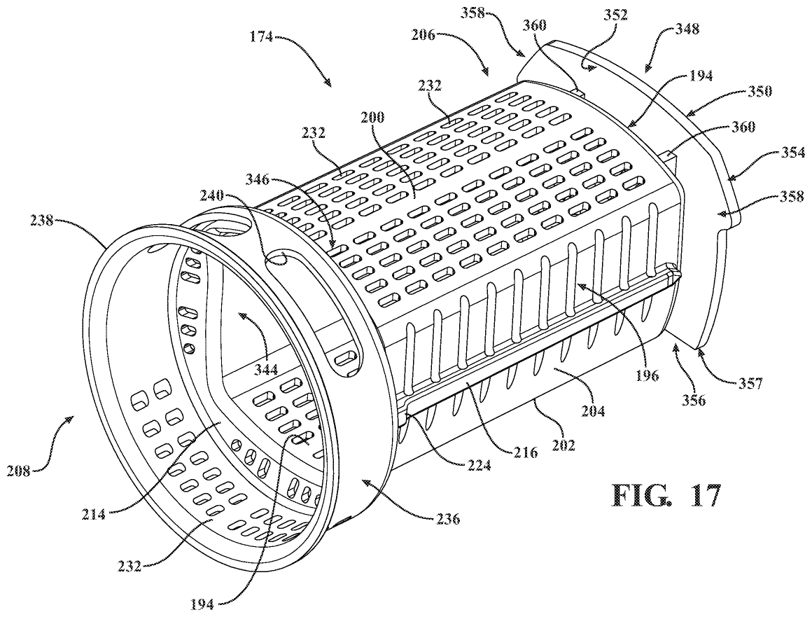

The base wall 194 of the basket 206 is arranged opposite the mouth 198 near a proximal end of the filter element 174. The base wall 194 is arranged nearer to the proximal end of the manifold 124 than the mouth 198 when the filter element 174 is disposed in the manifold volume 130. Certain configurations of the manifold 124 utilize a baffle wall 348 that is coupled to the filter element 174. More specifically, the baffle wall 348 is coupled to the basket 206 near a proximal end and spaced from the base wall 194. As shown in FIGS. 17-19, the base wall 194 may comprise at least one post 360 coupled to an outer surface of the base wall 194 and to the distally directed surface 352 of the baffle wall 348. Here, the at least one post 360 takes the form of two rails, each having a rectangular profile, that extend between the base wall 194 and the baffle wall 348 to space the baffle wall from the base wall 194. As will be discussed below in connection with the suction path 122, the space between the base wall 194 and the baffle wall 348 provides a greater surface area of the filter element 174 through which the waste material can flow while collecting the semisolid or solid waste material. In alternative to the rails, the at least one post 360 may comprise protrusions with any suitable profile that extend between the base wall 194 and the baffle wall 348 to couple the baffle wall 348 to the basket 206.

The filter element 174 may be utilized to position the seal 282 to block egress of fluid through the outlet opening 242. In certain implementations, the filter element 174 may include a seal retaining element (not shown) coupled to the basket 206. In particular, the seal retaining element may extend proximally from the base wall 194 of the basket 206. The seal retaining element may include axial profile generally shaped to the first leg 244 of the housing 128. The seal 282 may be coupled to the seal retaining element, for example, compressed between the rim of the seal retaining element and the rim 276 defining the outlet opening 242. The basket 206 may be disposed in the body portion 210 and the seal retaining element disposed in the first leg 244. Further structure and function of the seal retaining element and corresponding seal is described in the aforementioned U.S. application Ser. No. ______, filed Nov. 11, 2019. In implementations with the seal retaining element, the baffle wall 348 may be coupled to the seal retaining element. For example, the seal retaining element and the baffle wall 348 may be integrally formed with the seal retaining element extending through the baffle wall 348 in the proximal-to-distal direction.

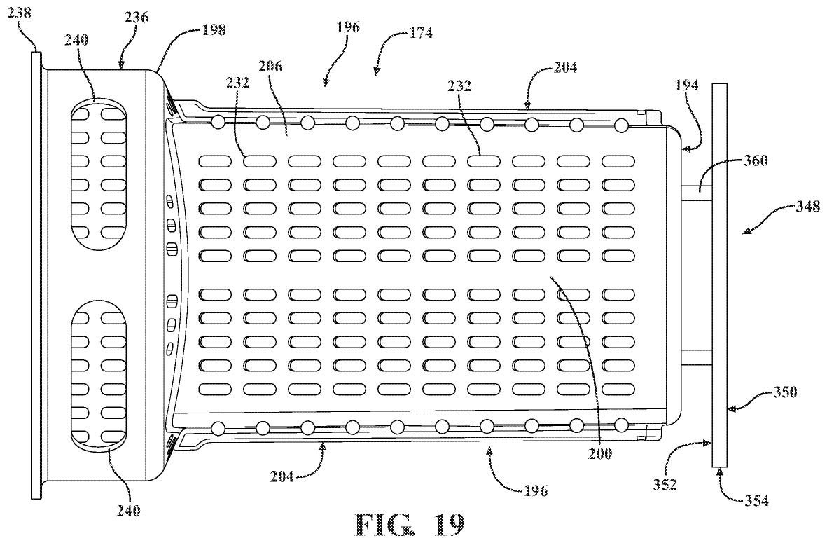

To facilitate coupling and locating the filter element 174 within the trunk 134, the filter element 174 may include at least one guide 216. With continued reference to FIGS. 17-19 and further reference to FIG. 8, the guide(s) 216 may include a rail extending laterally outward from one of the opposing sidewalls 204 of the basket 206 and oriented in the proximal-to-distal direction. The rail may be sized and oriented to be slidably inserted within a complementary slot defined between parallel railings extending laterally inward from the interior surface 131 of the body portion 210 of the trunk 134. FIGS. 8 and 19 shows two rails extending laterally outward from the opposing sidewalls 204, and two complementary slots extending laterally inward from opposing inner surfaces of the body portion 210. The guide(s) 216 include a proximal end near the base wall 194 of the basket 206, and a distal end adjacent or on the flared wall 214 of the brim 208. A laterally-outward taper 224 near the distal end of the guide(s) 216 may be configured to be in an interference arrangement with a complementary structure of the trunk 134 when the filter element 174 is fully seated within the manifold volume 130. The guide(s) 216 ensure the filter element 174 is fully seated within the manifold volume 130 with minimal “play” (e.g., inadvertent proximal, distal, lateral, and/or rotational movement from component tolerances or the like). Further, the guide(s) 216, in view of their relative shapes, dimensions, and/or positions, may prevent an unauthorized filter element from being coupled with the trunk 134, for example, during attempted reprocessing of the manifold 124.

The apertures of the filter element 174 may be shaped as holes 230 or pores 232, among others. The holes 230 and the pores 232 may be defined within any one or more of the base wall 194, the upper wall 200, the lower wall 202, the opposing sidewalls 204, the flared wall 214, and the brim 208. FIGS. 17-19 show the holes 230 defined within the base wall 194, the pores 232 defined within the base wall 194, the upper wall 200, the lower wall 202, the flared wall 214, and the brim 208. The apertures—in type and position—are arranged in a manner to minimize clogging of the filter element 174. During use, the amount of solid and semisolid waste material collected will begin to reduce the cross-sectional area of the suction path 122 across the filter element 174. In order to prevent a loss of performance of the manifold 124, cross-sectional area of the suction path 122 across the filter element 174 is relatively large. More specifically, the cross-sectional area of the suction path 122 through the baffle port 356 is less than the cross-sectional area of the suction path 122 across the filter element 174.

Further, the filter element 174 may be implemented as a foam or composite member configured to allow fluid to pass therethrough while capturing or collecting the semisolid or solid waste material. It should be appreciated that not all configurations of the manifold require use of the filter element 174, and manifold designs that do not include a filter element are contemplated. Further, the filter element 174 may be disposed in a location separate from the manifold volume 130 that is in fluid communication with the outlet opening 242 of the manifold 124.

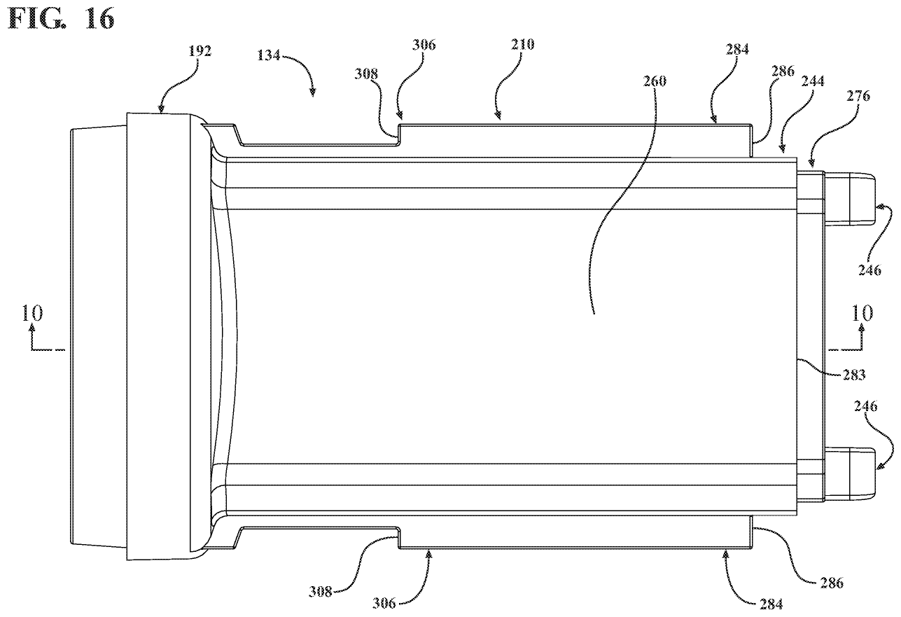

The brim 208 may include at least one sidewall 236 extending between the flared wall 214 and a distal rim 238 of the filter element 174. The sidewall 236 may be considered a singular side that is cylindrical in shape, or plural sides arranged in any suitable geometry. A length of the sidewall 236 may be less than the basket 206, and the sidewall 236 may include the outer diameter or dimension greater or larger than the outer diameter or dimension of the basket 206. The sidewall 236 may include the pores 232, particularly by a lower portion of the sidewall 236 as shown in FIG. 16. The sidewall 236 may further define at least one overfill opening 240 positioned on an upper portion of the sidewall 236. The overfill opening(s) 240 are configured to maximize the operational cycle of the manifold 124. Should a sufficient amount of the semisolid or solid waste material be generated over the course of the surgical procedure, an entirety of the basket 206 may become consumed with the accumulated semisolid or solid waste material. In other words, most or all of the holes 230 and the pores 232 of the filter element 174 may become clogged with the semisolid or solid waste material thereby restricting further flow. The overfill opening(s) 240 are sized and positioned to permit the suction path to be routed through the overfill opening(s) 240 and external to the basket 206. In other words, owing to understood principles of fluid dynamics where fluid assumes the path of least resistance, the suction path in the aforementioned scenario extends from the inlet bore(s) 138, through the head 132, through the overfill opening(s) 240, within the trunk 134 between the basket 206 and the inner surface of the trunk 134, and to the outlet opening 242. Further, the cavity 152 of the cap 140 defining a portion of the manifold volume 130 may afford additional volume distal to the filter element 174 for the accumulation of additional semisolid or solid waste material as the suction path is directed through the overfill opening(s) 240.

It is readily appreciated that the filter element 174 may be one of the aforementioned components of the manifold 124 that includes intricate geometries (e.g., the holes 230 and the pores 232) that become contaminated with the waste material during operation of the medical waste collection system 100. Thus, efforts to reprocess a previously used manifold may require removing the filter element 174, and thereafter cleaning and replacing the filter element 174 or inserting another, unused filter element. As a result, limiting removal of the filter element 174 is at least one advantageous manner in which the manifold 124 may discourage reprocessing of the same.

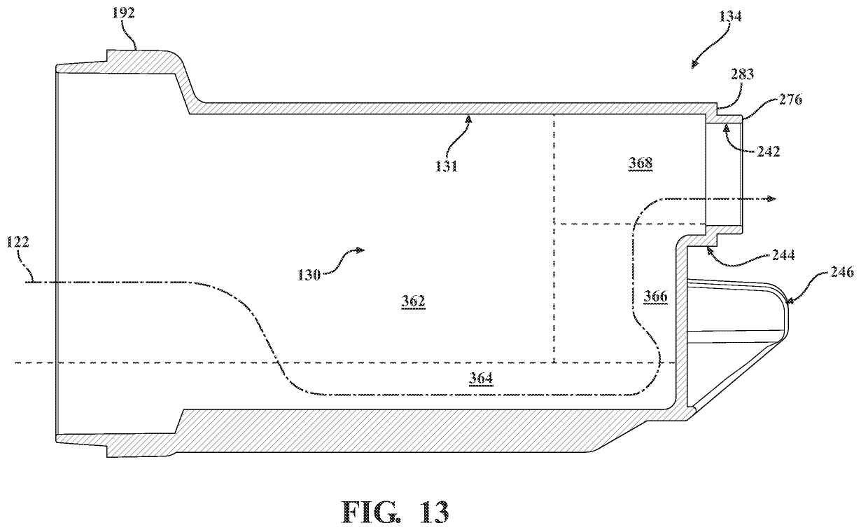

With renewed reference to FIGS. 7 and 13, where FIG. 7 shows a cross-sectional view of taken along line 7-7 in FIG. 16 the housing 128, the filter element 174, and the baffle wall 348, and FIG. 13 shows a simplified cross-sectional view of the manifold volume 130 and several regions thereof, with certain features removed for clarity. The manifold volume 130 comprises a filter region 362, a sump region 364, an outlet region 366, and a leg region 368, which cooperate to form the suction path 122. The suction path 12 is the path that the waste material follows through the manifold volume 130 in response to the negative pressure differential, or vacuum, between the inlet fitting 126 and the outlet opening 242.

As previously explained, as the waste material is removed during the surgical procedure it flows along the suction path 122 from the suction tube 120 through the inlet bore(s) 138, into the manifold volume 130, and out of the outlet opening 242. With respect to the manifold 124, and more specifically the manifold volume 130, the suction path 122 includes the filter region 362, the sump region 364, the outlet region 366, and the leg region 368. Broadly, the filter region 362 comprises the portion of the suction path 122 or the manifold volume 130 that is upstream, or encountered by the waste material first, of the filter element 174. Further, the sump region 364 broadly comprises the portion of the suction path 122 or the manifold volume 130 that is immediately downstream of the filter element 174 or below the uppermost aspect 357 of the baffle port 356. The outlet region 366 broadly comprises the portion of the suction path 122 or the manifold volume 130 that is between the uppermost aspect 357 of the baffle port 356 and below the lowermost aspect 250 of the outlet opening 242, and downstream of the baffle port 356. The leg region 368 broadly comprises the downstream portion of the suction path 122 or the manifold volume 130 above the lowermost aspect 250 of the outlet opening 242.

In the filter region 362, semisolid or solid waste material is collected and accumulates in the basket 206 under the influence of the vacuum. Owing to the direction of the suction path 122 (i.e., in the proximal direction), the semisolid or solid waste material generally accumulates on the base wall 194 of the basket 206, with fluid waste material that flows through the pores 232 and/or holes 230 in the base wall 194 being redirected by the baffle wall 348 toward the sump region 364.

After the semisolid or solid waste material has been collected by the filter element 174 the remaining fluid waste material generally comprises liquid waste, which may be entrained in and/or with air, as well as particles too small to be collected by the filter element 174. Continued influence of the vacuum on the manifold volume 130 motivates this fluid waste material in the sump region 364 to progress along the suction path 122 toward the baffle port 356. The ratio of liquid waste to air may vary throughout the duration of the surgical procedure as well as the procedure being performed. In one example, where a majority of the flow of waste material comprises air, droplets of liquid waste may tend to exist in suspension throughout the duration of the suction path 122. In another example with a greater ratio of liquid waste, the liquid waste may become separated from the air and begin to collect in the sump region 364 due to the influence of gravity while the air continues to flow through the baffle port 356. As the amount of liquid waste collected increases, the level of liquid waste in the sump region 364 will rise until such point that the liquid waste is at a height near the uppermost aspect of the baffle port 356. When the liquid waste is at or above this height, the vacuum pressure will lift the liquid waste out of the sump region 364 and into the outlet region 366, the leg region 368, and through the outlet opening 242.

When the manifold 124 has reached its capacity of semisolid or solid waste material or at the conclusion of the surgical procedure the source of vacuum is removed from the manifold 124 by removing the manifold 124 from the receiver 116, disabling the vacuum pump 110, or both. When the vacuum source is removed, liquid waste in the suction path 122 will remain in situ, including liquid waste in the leg region 368 and the outlet region 366. In the absence of the vacuum source, any residual liquid waste will tend to flow toward a point of lowest potential energy (i.e. “downhill”). Because the liquid waste was lifted into the outlet region 366 against the force of gravity, the arrangement of the outlet opening 242 near the upper aspect 252 of the housing 128 minimizes the amount of residual liquid waste that could flow toward the outlet opening 242 when the vacuum source is removed. By minimizing the amount of residual liquid waste that could flow toward the outlet opening 242 the likelihood of any residual liquid waste dripping or leaking out of the outlet opening 242 when the manifold 124 is removed from the receiver 116 is likewise reduced.

Referring again to FIG. 7, the upper wall 200 and the lower wall 202 of the basket 206 are spaced apart from the inner surface of the housing 128. In other words, a portion of the manifold volume 130 is external to the filter region 362. Further, the baffle wall 348 is spaced apart proximally from the base wall 194 to define a gap, and the upper aspect of the baffle wall 348 is abutting the interior surface 131 of the housing 128. A first suction path is established from the suction tube 120 to the outlet opening 242 across the lower wall 202 of the filter element 174, as previously explained. More specifically, the first suction path includes the waste material being moved under the influence of vacuum from the filter region 362, through the lower wall 202 into the sump region 364, the baffle port 356, the outlet region 366, the leg region 368, and the outlet opening 242. In such an arrangement, a second suction path may be established from the suction tube 120 to the outlet opening across the upper wall 202 of the filter element 174 and through the gap. More specifically, the second suction path includes the waste material being moved under the influence of vacuum from the filter region 362 and through the upper wall 200. The baffle wall 348 directs the second suction path downwardly through the gap between the base wall 194 and the baffle wall 348 into the sump region 364. The second suction path is effectively joined with the first suction path, and the waste material is moved through the baffle port 356, the outlet region 366, the leg region 368, and the outlet opening 242. The first and second suction paths may advantageously improve the flow characteristics of the manifold 124.

As previously mentioned, the rim 276 may have a width greater than a height such that the outlet opening 242 has a non-circular profile. In certain implementations, the baffle wall 348 is spaced apart from said housing to define the outlet region 366 (and the leg region 368), and the outlet region 366 is shaped to have a non-circular profile at least substantially similar to the non-circular profile as the outlet opening 242. Further, the leg region 368 may be shaped to have the non-circular profile at least substantially similar to the non-circular profile as the outlet opening 242. In other words, there may be at least a substantially constant non-circular profile from the baffle port 356 to the outlet opening 242. In such an arrangement, the suction path is associated with improved flow characteristics (e.g., less throttling or cavitation), and more fluid descends into the sump region 364 when the vacuum from the medical waste collection system 100 is terminated.

Several examples have been discussed in the foregoing description. However, the examples discussed herein are not intended to be exhaustive or limit the invention to any particular form. The terminology that has been used is intended to be in the nature of words of description rather than of limitation. Many modifications and variations are possible in light of the above teachings and the invention may be practiced otherwise than as specifically described.

1. A manifold for filtering medical waste including fluid received from a suction tube under influence of a vacuum provided by a medical waste collection system into which said manifold is configured to be inserted in a proximal direction and removed in a distal direction opposite the proximal direction, wherein the medical waste collection system includes a receiver defining an opening, said manifold comprising:
a housing defining a manifold volume and an outlet opening in fluid communication with said manifold volume, said housing comprising a rim defining said outlet opening positioned near an upper aspect of said housing when said manifold is oriented for insertion into the opening of the receiver;
a seal coupled to said rim, said seal comprising a seal body shaped to cover said outlet opening, said seal configured to prevent egress of the fluid when said manifold is removed from the receiver;
a filter element disposed within said manifold volume;
an inlet fitting configured to receive the suction tube such that a suction path is established from the suction tube to said outlet opening across said filter element; and
a baffle wall disposed within said manifold volume, wherein said baffle wall and said housing define a sump region of said manifold volume that is positioned below a lowermost aspect of said seal such that the fluid in said suction path is pulled from below said seal, thereby reducing a level of the fluid retained within said manifold.

2. The manifold of claim 1, wherein said housing further comprises a body portion having an upper wall, a lower wall, and opposing sides each extending between said upper and lower walls when said manifold is oriented for insertion into the opening of the receiver, and a proximal wall at least partially extending between said opposing sides, wherein said proximal wall and said baffle wall cooperate to define said sump region of said manifold volume.

3. The manifold of claim 2, wherein said housing further comprises a leg extending proximally from said proximal wall and wherein said rim is arranged on said leg.

4. The manifold of claim 2, further comprising a basket having a base and comprising said filter element, wherein said baffle wall is coupled to said basket and spaced from said base.

5. The manifold of claim 4, wherein said baffle wall comprises a wing portion and said opposing sides define a shoulder portion of said housing, wherein said wing portion and said shoulder portion have complementary profiles with said shoulder portion configured to receive said wing portion such that rotation of said basket is prevented when said filter element is disposed in said housing.

6. The manifold of claim 2, wherein said outlet opening is at least partially defined by said upper wall.

7. The manifold of claim 1, wherein said housing comprises a head and a body portion, said body portion having an interior surface at least partially defining said manifold volume, said head coupled to said inlet fitting and engageable with said body portion such that the suction tube and said manifold volume are in fluid communication.

8. The manifold of claim 1, wherein said baffle wall defines a baffle port arranged in said sump region, said baffle port configured such that an uppermost aspect of said baffle port is below said lowermost aspect of said seal when the manifold is inserted in the receiver.

9. The manifold of claim 8, wherein a cross-sectional area of said suction path through said baffle port is less than a cross-sectional area of said suction path across said filter element.

10. A manifold for filtering medical waste including fluid received from a suction tube received from a suction tube under influence of a vacuum provided by a medical waste collection system into which said manifold is configured to be inserted in a proximal direction and removed in a distal direction opposite the proximal direction, wherein the medical waste collection system includes a receiver defining an opening, said manifold comprising:
a housing defining a manifold volume and an outlet opening in fluid communication with said manifold volume, said housing comprising a body portion extending to a distal aspect, a leg extending proximally from said distal aspect and comprising a rim defining said outlet opening;
a seal coupled to said rim, said seal comprising a seal body shaped to cover said outlet opening, said seal configured to prevent egress of the fluid when said manifold is removed from the receiver;
a filter element disposed within said manifold volume;
an inlet fitting configured to receive the suction tube such that a suction path is established from the suction tube to said outlet opening across said filter element; and
a baffle wall disposed within said manifold volume and spaced apart from said distal aspect such that said baffle wall, and said distal aspect cooperate to reduce a cross-sectional area of said suction path near said outlet opening to less than a cross-sectional area of said suction path opposite said baffle wall.

11. The manifold of claim 10, wherein said body portion further comprises an upper wall, a lower wall, and opposing sides each extending between said upper and lower walls, and wherein said outlet opening is positioned near said upper wall when said manifold is oriented for insertion into the opening of the receiver.

12. (canceled)

13. The manifold of claim 11, further comprising a basket having a base and comprising said filter element, wherein said baffle wall is coupled to said base.

14-15. (canceled)

16. The manifold of claim 10, wherein said baffle wall defines a baffle port having an uppermost aspect, said baffle port configured such that said uppermost aspect is below a lowermost aspect of said seal when the manifold is inserted in the receiver.

17. A manifold for filtering medical waste including fluid received from a suction tube received under influence of a vacuum provided by a medical waste collection system into which said manifold is configured to be inserted in a proximal direction and removed in a distal direction opposite the proximal direction, wherein the medical waste collection system includes a receiver defining an opening, said manifold comprising:
a housing defining a manifold volume and an outlet opening in fluid communication with said manifold volume, said housing comprising a rim defining said outlet opening;
a filter element comprising a basket having a base, said filter element defining a sump region of said manifold volume between said base and said outlet opening; and
an inlet fitting configured to receive the suction tube such that a suction path is established from the suction tube to said outlet opening across said filter element; and
a baffle wall coupled to said base of said basket, said baffle wall arranged to redirect said suction path.

18. (canceled)

19. The manifold of claim 17, wherein said housing further comprises a body portion having an upper wall, a lower wall, and opposing sides each extending between said upper and lower walls when said manifold is oriented for insertion into the opening of the receiver, and a proximal wall at least partially extending between said opposing sides, wherein said proximal wall and said baffle wall cooperate to define said sump region of said manifold volume.

20-22. (canceled)

23. The manifold of claim 17, wherein said housing comprises a head and a body portion, said body portion having an interior surface at least partially defining said manifold volume, said head coupled to said inlet fitting and engageable with said body portion such that the suction tube and said manifold volume are in fluid communication and wherein said baffle wall comprises a perimeter surface configured to engage said interior surface of said body portion.

24. (canceled)

25. The manifold of claim 24, wherein said baffle wall defines a baffle port arranged in said sump region, said baffle port configured such that an uppermost aspect of said baffle port is below a lowermost aspect of said outlet opening when the manifold is inserted in the receiver.

26. The manifold of claim 25, wherein said baffle port is defined between said perimeter surface of said baffle wall and said interior surface of said body portion.

27. The manifold of claim 25, wherein a cross-sectional area of said suction path through said baffle port is less than a cross-sectional area of said suction path across said filter element.

28. The manifold of claim 17, wherein said sump region of said manifold volume is positioned below a lowermost aspect of said outlet opening such that the fluid in said suction path is pulled from below said outlet opening, thereby reducing a level of the fluid retained within said manifold.

29-38. (canceled)
